(19) United States
(12) Patent Application Publication
(10) Pub. No.: US 2007/0167338 A1
(43) Pub. Date: Jul. 19, 2007

(54) Multiphase personal care compositions comprising beads

(76) Inventors: Colin Michael McHugh, Mason, OH (US); Edward Dewey Smith III, Mason, OH (US); Karl Shiqing Wei, Mason, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/650,920

(22) Filed: Jan. 8, 2007

Related U.S. Application Data
(60) Provisional application No. 60/757,349, filed on Jan. 9, 2006.

(51) Int. Cl. A61K 8/00

(52) U.S. Cl. 510/130

(57) ABSTRACT

Multiphase personal care compositions that can deliver one or more benefits in a single product. The multiphase personal care compositions can provide multiple benefit effects through the inclusion of beads. The beads are stable and maintain their arrangement within the composition, withstanding the conditions reasonably expected during shipment, storage, and use. The compositions comprise a plurality of visually distinct phases wherein one of the phases comprises one or more beads, where the beads are individually visually distinct and visible within the composition. Further, the phases are arranged in a non-random pattern. A method for making the multiphase personal care compositions is also disclosed.

### CROSS REFERENCE TO RELATED APPLICATION

This application claims the benefit of U.S. Provisional Application No. 60/757,349 filed Jan. 9, 2006.

### FIELD OF THE INVENTION

The invention relates to multiphase personal care compositions comprising at least two visually distinct phases. At least one phase comprises one or more visually distinct beads.

### BACKGROUND OF THE INVENTION

Personal care compositions that involve more than one functional component or even more than one phase are known. These compositions have been employed to cleanse, moisturize, deliver actives, hide imperfections, alter the color of, condition, shine, and refresh skin and hair. However, there still remains a need for single compositions which are not only highly functional and can impart multiple benefits, but are also aesthetically pleasing.

Early attempts at providing products with multiple phases employed multi-chamber packaging. These multi-chamber packages would contain, for example, separate cleansing and conditioning products that remained physically separated to maintain stability during prolonged storage times. These packages were designed to simultaneously dispense the products in order to effectuate a dual cleansing and conditioning effect. In other attempts, the cleaning and conditioning products were mixed prior to dispensing in order to effectuate the dual cleansing and conditioning effect.

Although such dual-chamber delivery systems provided a limited degree of improved convenience sought by consumers, they often failed to provide consistent and uniform performance because of the uneven dispensing of the different phases that can be inherent in such a dual-chamber system. In particular, one product could be emptied through use long before the other chamber was exhausted. This would result in wasted, unused product, a visually displeasing container, and sub-optimal product performance. These issues often lead to displeasure and frustration amongst consumers.

Compositions including spheres or beads randomly dispersed throughout the composition are also well known and widely used. However, during shipment and usage by the consumer, the beads can migrate throughout the composition and create a less-than-ideal experience for the consumer. For instance, the beads can aggregate to the bottom of the container, leaving the consumer with variable results during use.

While the compositions and disclosures of the prior art provide useful advances in the art of multiphase compositions, there remains the need for improved compositions that can deliver one or more benefits in a single product. Furthermore, there remains the need for a product that can provide multiple benefit effects through the inclusion of beads where the beads maintain their original aesthetically pleasing arrangement within the composition, withstanding the conditions reasonably expected during shipment, storage, and use.

### BACKGROUND ART

The following references relate to compositions having multiple phases or beads: U.S. Pat. No. 6,773,811, issued Aug. 10, 2004, in the name of Ferguson et al.; U.S. Pat. No. 6,797,683, issued Sep. 28, 2004, in the name of Shana'a et al.; U.S. Pat. No. 6,294,179, issued Sep. 25, 2001, in the name of Lee et al.; U.S. Pat. No. 6,255,264, issued Jul. 3, 2001, in the name of Fleurot et al.; U.S. Pat. No. 6,533,873, issued Mar. 18, 2003, in the name of Margosiak et al.; U.S. Pat. No. 5,011,690, issued Apr. 30, 1991, in the name of Garvey et al.; U.S. Pat. No. 6,780,826, issued Aug. 24, 2004, in the name of Zhang et al.; U.S. Pat. No. 6,673,371, issued Jan. 6, 2004, in the name of Brown et al.; U.S. Pat. No. 6,764,991, issued Jul. 20, 2004, in the name of Puvvada et al.; U.S. Pat. No. 6,362,156, issued Mar. 26, 2002, in the name of Hsu et al.; U.S. Pat. No., 6,051,541, issued Apr. 18, 2000, in the name of Neuser et al.; U.S. Pat. No. 5,661,189, issued Aug. 26, 1997, in the name of Grieveson et al.; U.S. Pat. No. 4,159,028, issued Jun. 26, 1979, in the name of Barker et al.; U.S. Pat. No. 4,335,103, issued Jun. 15, 1982, in the name of Barker et al.; U.S. Pat. No. 6,245,344, issued Jun. 12, 2001 in the name of Thibiant et al.; U.S. Pat. No. 6,367,519, issued Apr. 9, 2002, in the name of Thibiant et al.; U.S. Pat. No. 6,516,838, issued Feb. 11, 2003, in the name of Thibiant et al.; U.S. 2004-0223991, published Nov. 11, 2004, in the name of Wei et al.; U.S. Pat. No., 4,966,205, issued Oct. 30, 1990, in the name of Tanaka et al.

### SUMMARY OF THE INVENTION

The present invention provides multiphase personal care compositions that can deliver one or more benefits in a single product. The multiphase personal care compositions can provide multiple benefit effects through the inclusion of beads. The beads are stable and maintain their arrangement within the composition, withstanding the conditions reasonably expected during shipment, storage, and use. The compositions comprise a plurality of visually distinct phases wherein one of the phases comprises one or more beads, where the beads are individually visually distinct and visible within the composition. Further, the phases are arranged in a non-random pattern.

In one embodiment of the multiphase personal care composition, the difference in density between each of two phases is less than about 0.15 g/cm³. In another embodiment, the multiphase personal care composition comprises beads that are from about 0.1 mm to about 10 mm in size. Although the personal care composition can be in any suitable form, in particular embodiments, the personal care composition can be a body wash or a lotion.

### DETAILED DESCRIPTION OF THE INVENTION

The current invention relates to a composition comprising at least two visually distinct phases wherein one of the phases comprises one or more beads, where the beads are individually visually distinct, and stable within the product container. Further, the two phases are strategically arranged in a non-random pattern.

The term “bead” as used herein, unless otherwise specified, refers to a particle which is visually distinct. A bead, as used herein, is about 0.1 mm and larger. The bead can be of any size, shape, or color, according to the desired characteristic of the product, so long as it is distinctively detected as an individual particle by the naked eye such that a person of unaided, 20/20 vision would recognize an individual bead from a distance of 1 ft.

The term “ambient conditions” as used herein, unless otherwise specified, refers to surrounding conditions at one (1) atmosphere of pressure, 50% relative humidity, and 25° C.

The term “personal care composition” as used herein, unless otherwise specified, refers to compositions for topical application to the skin or hair. Such personal care compositions can include, but are not limited to, body washes, shampoos, conditioners, hair styling products, cleansers, soaps, cosmetics, foundations, antiperspirants, deodorants, moisturizers, lotions, creams, ointments, skin-moisturizers, skin-conditioners, combinations thereof, and the like.

The term “stable” as used herein, unless otherwise specified, refers to compositions that maintain at least two “separate” phases when in physical contact at ambient conditions for a period of at least 180 days.

The term “separate” as used herein, is meant that there is substantially no mixing, observable to the naked eye, such that a person of unaided, 20/20 vision would recognize the separate phases from a distance of 1 ft.

The term “phase” as used herein refers to a homogeneous, physically distinct, and mechanically separable portion of matter present in a non-homogeneous physical-chemical system. The term “visually distinct phase” or “visually distinctive phase” or “visually discernable phase” as used herein, refers to a region of the multiphase personal care composition having one average composition, as distinct from another region having a different average composition, wherein the distinction can be made by a visual inspection. The distinction can be made by the naked eye such that a person of unaided, 20/20 vision would recognize the distinction from a distance of 1 ft.

This would not preclude the distinct regions from comprising two similar phases where one phase could comprise pigments, dyes, particles, and various optional ingredients, hence a region of a different average composition. A phase generally occupies a space or spaces having dimensions larger than the colloidal or sub-colloidal components it comprises. A phase may also be constituted or re-constituted, collected, or separated into a bulk phase in order to observe its properties, e.g., by centrifugation, filtration or the like.

The term “multiphase” or “multiple-phase” as used herein, means that the phases of the present compositions occupy separate but distinct physical spaces inside the package in which they are stored, but are in substantially direct physical contact with one another (i.e., they are not separated by a barrier and they are not emulsified or mixed to any significant degree).

The term “non-random pattern” as used herein means that the phases are arranged in a predetermined pattern that remains substantially unaltered at ambient conditions for a period of at least 180 days. The lack of change to the predetermined pattern is determined by observation with the naked eye of a person of unaided, 20/20 vision from a distance of 1 ft.

The term “swirl” or “swirled” as used herein means that one or more phases in the composition occupies separate, but distinct, physical spaces inside the container in which it is stored, but is in direct contact with one another in three or more distinct layers.

The term “marble” or “marbled” as used herein refers to a swirled design with a veined and/or mottled appearance similar to marble.

As used herein “tottle” refers to a bottle which rests on its neck or mouth, from which its contents are filled and dispensed from. The neck or mouth end is also the end upon which the bottle is intended to rest or sit upon (e.g., the bottle's base) for storage by the consumer and/or for display on the store shelf.

A. Multiphase Composition

In one embodiment, the present invention provides a multiphase personal care composition comprising: (a) a first visually distinct phase that comprises one or more visually distinct beads; and (b) a second visually distinct phase. The first visually distinct phase and second visually distinct phase form a visually distinct, non-random pattern.

1. Beads

The multiphase personal care composition of the present invention can comprise at least one bead. The beads of the present invention can have a particle size (volume average based on the particle size measurement described herein) of greater than about 0.1 mm, preferably from about 0.1 mm to about 10 mm, more preferably from about 0.1 mm to about 5 mm, and even more preferably from about 0.1 mm to about 3 mm in size. Furthermore, the beads can be visually distinct.

The beads can have any suitable shape. For example, beads having a spherical, an oval, an irregular, or any other shape can be used herein. In one embodiment, the ratio of the largest dimension to the smallest dimension (defined as the Aspect Ratio) of the beads is less than about 12. More preferably, the Aspect Ratio of the beads is less than about 8, still more preferably the Aspect Ratio of the beads is less than about 5.

Any suitable type of bead can be used herein. For example, suitable beads can include natural, synthetic, semi-synthetic, or hybrid beads (e.g., synthetic beads can made of cross-linked or non cross-linked polymers), or mixtures thereof. Non-limiting examples of suitable beads can include, but are not limited to: Exfoliating beads, organogel beads, texturing beads, cleansing beads, surfactant beads, porous beads, crystalline beads, liposome beads, microcapsule beads, retinoid beads, polymeric beads, organic beads, oil beads, abrasive beads, fruit puree-beads, vitamin beads, and combinations thereof. Preferably, the multiphase personal care composition of the present invention comprises the bead at a functionally efficacious level. Beads that break, beads that do not break, beads that dissolve, or beads that do not dissolve upon application to the skin or hair can be used in a personal care composition.

Beads can be included in the personal care composition at any suitable desired level. In one embodiment, the beads are present from at least about 0.1% by weight of the composition, preferably at least about 0.2% by weight of composition, even more preferably at least about 0.5%, more preferably at least about 1%, and still more preferably at least 2% by weight of composition. In the multiphase personal care composition of the present invention, preferably the beads comprise less than about 50% by weight of composition, more preferably less than about 30%, still more preferably less than about 20%, and even more preferably less than about 10% by weight of composition.

Preferably, the beads will also have physical properties which are not significantly affected by typical processing of the composition. Preferably, the beads will have a melting point of greater than about 70° C., more preferably the beads will have a melting point of greater than about 80° C., and even more preferably the beads will have a melting point of greater than about 95° C. As used herein, melting point refers to the temperature at which the solid beads transition from a solid state to a liquid or fluid state or undergoes significant deformation or physical property changes.

In another embodiment, beads of present invention are cross-linked or have a cross-linked surface membrane. These beads do not exhibit a distinct melting point. Cross-linked particles can also be useful as long as they are stable under the processing and storage conditions used in the making of multiphase personal care compositions.

2. Phases

The multiphase personal care compositions of the present invention comprise one or more phases arranged in a non-random pattern. At least one phase comprises one or more visually distinct beads. Within the non-random pattern, a bead may be arranged randomly, or non-randomly, in relation to other beads. A phase can comprise any suitable material or composition. Non-limiting examples of a phase can include: a cleansing phase, a benefit phase, or a non-lathering structured aqueous phase.

In one embodiment, the multiphase personal care composition comprises two or more visually distinct phases. In a particular embodiment, a first phase has the same composition as a second phase, with the exception that the second phase comprises a colorant not present in the first phase. In another embodiment, a first phase comprises a different composition than a second phase.

The phases of the multiphase personal care composition can be arranged in any suitable non-random pattern. Suitable non-random patterns can include, but are not limited to, the following examples: swirled, marbled, rectilinear, interrupted striped, check, mottled, veined, clustered, speckled, geometric, spotted, ribbons, helical, striped, arrayed, variegated, textured, grooved, ridged, waved, sinusoidal, spiral, twisted, curved, cycle, streaks, striated, contoured, anisotropic, laced, weave or woven, basket weave, spotted, and tessellated. In particular embodiments, the pattern is selected from the group consisting of swirled, marbled, and combinations thereof.

3. Density

In particular embodiments, it is desirable for the density of the phases to be substantially matched in order to maintain the non-random pattern in the composition. Preferably, the density difference between one phase and another phase is less than about 0.15 g/cm³, more preferably the density difference is less than about 0.10 g/cm³, even more preferably the density difference is less than about 0.05 g/cm³, and still more preferably the density difference is less than about 0.01 g/cm³. If the density of the different phases is not substantially equal, the densities of the different phases may be adjusted before the phases are combined to form the multiphase personal care composition. Density may be adjusted by any suitable means.

For example, to adjust the density of a phase, low density microspheres can be added to the phase of the composition. The low density microspheres employed to reduce the overall density of a phase can be particles having a density less than about 0.7 g/cm³, preferably less than about 0.2 g/cm³, more preferably less than about 0.1 g/cm³, still more preferably less than about 0.05 g/cm³. The low density microspheres can generally have a diameter less than about 0.2 mm, preferably less than about 0.1 mm, and even more preferably less than about 0.04 mm. Such microspheres can be produced from any appropriate inorganic or organic material, compatible with use on the skin; that is, nonirritating and nontoxic. Preferably, the microspheres do not negatively impact the product performance.

Conversely, just as low density microspheres can be added to a phase of the present invention to modify density, high density materials can be added to a phase to increase its density. The high density particles employed to increase the overall density of one phase can be particles having a density greater than about 1.1 g/cm³, preferably greater than about 1.5 g/cm³, more preferably greater than about 2.0 g/cm³, and still more preferably greater than about 2.5 g/cm³. The high density particles can generally have a diameter less than about 0.2 mm, preferably less than about 0.1 mm, and still more preferably less than about 0.04 mm. In particular embodiments the high density particles are selected from water-insoluble inorganic materials, metals, metal oxides, metal alloys, and mixtures thereof. Non-limiting examples of high density particles can include calcium carbonate, silica, clays, mica, talc, iron, zinc, copper, lead, titanium dioxide, zinc oxide, combinations thereof, and the like.

4. Product Form

The multiphase personal care compositions of the present invention can be in any suitable desired product form. Furthermore, the multiphase personal care compositions of the present invention may be prepared by any known or otherwise effective technique suitable for making and formulating the desired multiphase product form. The multiphase personal care compositions can be, for example, in the form of a liquid, semi-liquid, cream, lotion or gel composition intended for topical application to hair or skin. Such compositions can include, but are not limited to, body washes, shampoos, conditioners, hair styling products, cleansers, soaps, cosmetics, foundations, anti-perspirants, deodorants, moisturizers, lotions, creams, ointments, or combinations thereof.

B. Product Container

Any suitable product container can be used herein. In one embodiment, the container is a bottle. In another embodiment, the container is a bottle with a flat cap. In still another embodiment, the container is a tottle. In a specific embodiment, a clear or opaque bottle is used. In a particular embodiment, a container having a clear or opaque region through which the multiphase personal care composition can be seen is used.

If a tottle is used, the personal care composition can be stored with the end from which it was filled facing downward. This avoids the need to tip the bottle over to dispense product upon each usage by the consumer, thus eliminating the movement of air bubble(s) through the length of the product in the package upon each usage occasion by the consumer. Movement of air bubble(s) through the length of the product is undesirable from an aesthetic standpoint, as this can disrupt the attractive appearance of a product having visually distinct phases. This loss of product attractiveness undesirably detracts from the consumer's enjoyment of the product. In yet another embodiment, the container can be a bottle that can stand on either end in an upright position.

The closure on a tottle can be flat or concave, such that the tottle, when stored, rests on the closure. Non-limiting examples of the closure include a cap, flip-top, screw-on, screw-on flip-top cap, rocker cap, and the like.

C. Filling the Empty Product Container to From a Container of Multiphase Composition

The multiphase personal care composition can be prepared by any suitable means. For example, filling the empty product container with the multiphase personal care composition can comprise transferring predetermined amounts of the different phases through at least one dispensing means into an empty product container. Additionally, the multiphase personal care compositions can be prepared by the method and apparatus as disclosed in U.S. Pat. No. 6,213,166 issued to Thibiant, et al. on Apr. 10, 2001. The method and apparatus allows two or more compositions to be filled with a spiral configuration into a single container. The method requires that at least two nozzles be employed to fill the container. The container is placed on a moving stage and spun as the composition is introduced into the container.

Alternatively, it is effective to combine at least two phases by first placing the separate compositions in separate storage tanks having a pump and a hose attached. The phases are then pumped in predetermined amounts into a single combining section. Next, the phases are moved from the combining sections into a blending section and the phases are mixed in the blending section. The next step involves pumping the product that was mixed in the blending section via a hose into a single nozzle, then placing the nozzle into a container and filing the container with the resulting product such that the single resulting product exhibits a visually distinct non-random pattern of the phases.

The shape and size of the particular product container used will dictate the rate of filling, rotation of the bottle, frequency of rotation of the bottle, and movement of the bottle during filling, as these factors can have a direct impact on the shape, size, and overall appearance of the pattern in the multiphase composition. The starting position of the bottle (proximity to the multiphase dispenser, as well as relative position when initially being filled), also affect the appearance of the pattern in the multiphase composition.

Preferably, minimal void volume remains in the finished container of multiphase composition. Minimizing the void volume can be accomplished through any suitable means. Such methods can include, but are not limited to, filling methods known in the art such as: (1) overfilling the container, followed by cleaning off the overfill; (2) topping off (e.g., by using a secondary filler, filling by hand, etc.); (3) using a container closure that displaces at least part of the void area (e.g., a deformable plug that occupies at least part of the void area); (4) utilizing a venting closure that allows the void (e.g., air bubble) to escape; and (5) carefully filling the container.

In one embodiment an empty product container is filled with the phases to form a container of multiphase personal care composition, optionally having a void volume of from about 0% to about 4%, in another embodiment optionally having a void volume from about 0% to about 3%, and in still another embodiment, optionally having a void volume from about 0% to about 2%.

Another effective method of combining multiple phases involves placing one or more compositions in separate vessels. Each vessel should be equipped with at least one outlet for dispensing the composition inside. Regarding the compositions, each vessel should contain a distinct composition and at least one of the compositions should comprise visually distinct beads. The composition from each vessel can be dispensed from the individual vessels and can be combined prior to dispensing from an outlet, or alternatively, the compositions can undergo some degree of mixing prior to being dispensed by being combined in a single common area, such as, but not limited to a pipe, static mixer, baffled area, etc. Alternatively, the compositions cay be dispensed into the empty product container through at least one dispensing means.

Placement of the dispensing means during filling of these multiple liquid phase products is an additional process variable. In one embodiment, the process involves initially placing the nozzle near the bottom of the container to be filled and lifting the dispensing means as the container fills. In other variations, the container itself can be raised on the dispensing means, or the container can be filled from the top. In one possible variation, the container can be filled upside down and the bottom attached to the container following filling.

These multiple-phase compositions are processed so that the two separate phases are in substantial direct physical contact, but remain stable and are arranged in a visually distinct non-random pattern. One or more of the phases is comprised of beads arranged either randomly or non-randomly with respect to the other beads within the non-random pattern.

D. Analytical Methods

1. Void Volume of Compositions

The void volume left in a package after the completion of the filling and capping operations is determined as follows:
a. Take a representative sampling of packages and closures, filling the package (manually if need be) with 25° C. water up to the point of overflow, and cap with the closures, drying off any water that is displaced. Record an average weight.
b. For the same package and closure, obtain an average weight of empty and dry package and closure.
c. Fill a representative number of packages with the intended composition, preferably deaerated, capping with closures. Obtain an average weight.

d. Calculate the void volume (this will correspond in most cases to the air bubble that will travel through the package when the package is inverted from its original filling orientation):

| | |
|---|---|
| Water Volume | (Water Weight Ave. - Empty Weight Ave.)/ |
| | Water Specific Gravity |
| Composition | (Comp. Weight Ave. - Empty Weight Ave.)/ |
| Volume | Comp. Specific Gravity |
| Void Volume | 100 * (Water Volume - Composition Volume)/ |
| % | Water Volume |

Note:
use the same weight units for each weight measurement.

2. Bead-Size Measurement Method

The bead size measurement method is typical of those known in the art, and utilizes a standard Nikon optical microscope, with standard transmitted light using ×10 objective. To aid accuracy, Lucia G software (by Nikon) is used with the following procedure. The first step of analysis requires the user to scan and select a field that is representative of the bulk—this typically requires multiple preparations for accuracy. The observed image is transmitted via JVC video camera to a standard monitor and each bead is measured by using the standard Measure macro; namely, clicking on each side of the bead—hence measuring a diameter. To account for none spherical beads, the ‘diameter’ is always assessed horizontally across the monitor. By measuring in one plane, the technique automatically compensates for non spherical geometry and due to the large number of beads measured results in an equivalent average diameter. Although equivalent diameters may be determined by measuring the major and minor axes and calculating equivalent diameter via aspect ratio equations, the above technique provides equally accurate results.

Since it is typical human nature to count the largest beads first and thus to ensure that all beads are counted and measured, a small (typically using an erasable pen) dot should be placed on the monitor over each counted bead. The count procedure is continued until every single visible bead is counted within the field. In the case of a very small bead size distribution, this may result in over 400 counts. In the case of larger bead sizes, one might expect approximately 100 counts per field, however in such cases additional fields are selected to ensure at least 200 separate beads are counted. In summary, in all cases at least 200 separate beads should be measured and in all cases all beads (in practice the upper limit being 400-500) in one field are counted. On average, across all the examples sighted herein, about 300 beads should be measured per sample. Analysis can be standard volume average calculated by hand to demonstrate the technique or, more typically, using the standard Measure macro that automatically sorts the data reporting a volume average (assuming a spherical geometry based on the diameter measured above).

3. Density Measurement

Fluid density is obtained using a graduated cylinder to measure a volume of 100 mL of a phase. An electronic balance is used to measure weight per standard laboratory practice, to within 0.001 grams/cm³ (g/cm³) fluid density.

4. Particle Density Method

The test is run at ambient conditions. First, a representative particle or group of particles is obtained. A first fluid is selected, having a density as measured above. The particle(s) is transferred into the fluid and centrifuged for 2 minutes at 2500 rpm on a laboratory centrifuge, e.g., Heraeus Labofuge model 400. If the particle(s) is observed to sink, it is denser than the fluid density, and a second fluid is obtained having a density higher than the first fluid. A representative particle is transferred into the second fluid, and is centrifuged under the same conditions. If the particle floats in the first fluid, a representative second fluid is obtained which has a lower density than the first fluid, and a representative particle is transferred into the second fluid, and is centrifuged under the same conditions. If the particle is neutrally buoyant in the first fluid, the particle has the same density as the first fluid, in g/cm³. If the particle sinks in the second fluid, a third fluid is obtained having a density higher than the second fluid, and a representative particle is transferred into the third fluid, and then centrifuged under the aforementioned conditions. If the particle floats in the second fluid, a fluid is selected having a lower density than the second fluid, a representative particle is transferred into the third fluid, then centrifuged under the aforementioned conditions. If the particle is neutrally buoyant in the second fluid after 2 minutes of centrifuging, the particle has the same density as the second fluid. The test is continued by continuing to select

Fluids are selected from common, non-thickened laboratory materials including alcohols, linear hydrocarbons, branched hydrocarbons, ethoxylated. alcohols, water, polyols such as glycerine and propylene glycol, silicone fluids, fluorinated materials, and miscible mixtures thereof. For particles having a density higher than (i.e., greater than about 2 g/cm³) organic laboratory materials, e.g. some pigments, an alternate method standard for high density materials such as a gas pycnometer can be used. For materials having a density lower than common laboratory fluids, i.e., less than about 0.785 g/cm³, gas pycnometry can be used. Fluids are selected which do not significantly absorb into or desorb or solubilize components of the particle for the duration of the test.

### E. EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Examples 1-4

The following are non-limiting examples of an in-shower body wash/lotion product according to the present invention.

| | |
|---|---|
| | Amount (By Weight |
| Ingredients | of First Phase) |

FIRST PHASE (EX. 1-4)

| | |
|---|---|
| Petrolatum ^{a} | 22.0% |
| Diisopropyl Sebacate ^{b} | 3.5% |
| Hydroxypropyl Starch Phosphate ^{c} | 3.5% |
| Stearyl Alcohol, Cetyl Alcohol, and | 2.4% |
| Polysorbate 60 blend ^{d} | |
| Fragrance | 1.2% |
| Preservative ^{e} | 0.293% |
| Phenoxyethanol | 0.25% |
| Disodium EDTA ^{f} | 0.12% |
| Water | Balance to 100% |

| | |
|---|---|
| | Amount (By Weight |
| Ingredient | of Second Phase) |

SECOND PHASE (EX. 1)

| | |
|---|---|
| Colored oxidized polyethylene beads ^{g} | 100% |

SECOND PHASE (EX. 2)

| | |
|---|---|
| Colored polyethylene beads ^{g} | 100% |

SECOND PHASE (EX. 3)

| | |
|---|---|
| Mannitol | 68.5% |
| Cellulose | 25% |
| Colorants | 3% |
| Vitamins | 3% |
| Hydroxypropyl methylcellulose | 0.5% |

SECOND PHASE (EX. 4)

| | |
|---|---|
| Large (>1 mm) Petrolatum beads | 100% |

^{a} Commercially available from Crompton Witco under the tradename G-2180 Petrolatum ™.
^{b} Commercially available from Noveon under the tradename SCHERCEMOL DIS ™.
^{c} Commercially available from National Starch under the tradename STRUCTURE XL ™.
^{d} Commercially available from Croda under the tradename POLAWAX Pastilles ™.
^{e} Commercially available from Lonza under the tradename GLYDANT PLUS Liquid ™.
^{f} Commercially available from Akzo Nobel under the tradename DISSOLVINE NA2-S ™.
^{g} Commercially available from Accutech under the tradename ACCUSCRUB ™.

The first and second phases of the multiphase personal care composition exemplified above are both opaque. The first and second phases are combined as described below and form a visually distinct pattern wherein the pattern is a swirl.

The multiphase personal care composition exemplified above, which is an in-shower body lotion, is made by separately making the first phase by conventional methods as known in the art, then adding the second phase, and then combining them according to the process described in US 2004/0219119 A1 to form the finished multiphase personal care composition.

The first and second phases are combined to form a multiphase personal care composition according to a process similar to that described in US 2004/0219119 A1. The process is also similar to that described in U.S. Pat. No. 4,159,028. The first phase is pumped from the first storage tank into a receiving cavity. The second phase is pumped from the second storage tank into the same receiving cavity. The first and second phases (approximately equal amounts) are then pumped out of the receiving cavity and through a filling nozzle to form the multiphase personal care composition. A plastic bottle, or other package, is placed directly underneath the filling nozzle to receive the multiphase personal care composition from the filling nozzle. The plastic bottle is positioned on a bottle holding stand that lowers and rotates the bottle during filling. As the multiphase personal care composition flows from the filling nozzle, the bottle holding stand lowers and rotates the bottle during filling at about 250 rpm. When the bottle is filled with the multiphase personal care composition, the process is complete. The phases in the multiphase personal care composition form a visually distinct striped pattern.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A multiphase personal care composition comprising:
a) a first visually distinct phase comprising one or more visually distinct beads; and
b) a second visually distinct phase;
wherein said first visually distinct phase and said second visually distinct phase form a visually distinct non-random pattern.

2. The multiphase personal care composition of claim 1 wherein said first visually distinct phase and said second visually distinct phase are in physical contact.

3. The multiphase personal care composition of claim 1 wherein said phases are stable.

4. The multiphase personal care composition of claim 1, wherein said multiphase composition has a difference in density between each of said phases of less than about 0.15 g/cm³.

5. The multiphase personal care composition of claim 1, wherein said multiphase composition has a difference in density between each of said phases of less than about 0.1 g/cm³.

6. The multiphase personal care composition of claim 1, wherein said multiphase composition has a difference in density between each of said phases of less than about 0.05 g/cm³.

7. The multiphase personal care composition of claim 1, wherein said multiphase composition has a difference in density between each of said phases of less than about 0.01 g/cm³.

8. The multiphase personal care composition of claim 1, wherein said visually distinct beads are arranged randomly within said visually distinct non-random pattern.

9. The multiphase personal care composition of claim 1, wherein said visually distinct beads are arranged non-randomly within said visually distinct non-random pattern.

10. The multiphase personal care composition of claim 1, wherein said beads are from about 0.1 mm to about 10 mm in size.

11. The multiphase personal care composition of claim 1, wherein said beads are from about 0.1 mm to about 5 mm in size.

12. The multiphase personal care composition of claim 1, wherein said beads are from about 0.1 mm to about 3 mm in size.

13. The multiphase personal care composition of claim 1, wherein said non-random pattern is selected from the group consisting of swirled, marbled, striped, rectilinear, interrupted striped, checked, mottled, veined, clustered, speckled, geometric, spotted, ribbons, helical, arrayed, variegated, textured, grooved, ridged, waved, sinusoidal, spiral, twisted, curved, cycle, streaked, striated, contoured, anisotropic, laced, weaved or woven, basket weave, spotted, tessellated, and combinations thereof.

14. The multiphase personal care composition of claim 1, wherein said non-random pattern is marbled.

15. The multiphase personal care composition of claim 1, wherein said non-random pattern is swirled.

16. The multiphase personal care composition of claim 1, wherein said bead is selected from the group consisting of exfoliating beads, organogel beads, texturing beads, cleansing beads, surfactant beads, porous beads, crystalline beads, liposome beads, microcapsule beads, retinoid beads, polymeric beads, organic beads, oil beads, abrasive beads, fruit puree beads, vitamin beads, and combinations thereof.

17. The multiphase personal care composition of claim 1, wherein said multiphase personal care composition is a body wash.

18. The multiphase personal care composition of claim 1, wherein said multiphase personal care composition is a lotion.

19. A stable multiphase body wash composition comprising:
a) a first visually distinct phase comprising one or more beads of from about 0.1 mm to about 3 mm in size, wherein said beads are arranged randomly throughout said first visually distinct phase; and
b) a second visually distinct phase wherein said second visually distinct phase is in physical contact with said first visually distinct phase;
wherein, said body wash composition has a difference in density between said first visually distinct phase and said second visually phase of less than about 0.01 g/cm³, and said first visually distinct phase and said second visually distinct phase form a swirl pattern.

20. A process for making a visually distinct, multiphase personal care composition comprising the steps of:
a) placing one or more phases in separate vessels equipped with at least one dispensing means for transferring said phases from said vessels, wherein at least one phase comprises visually distinct beads;
b) providing an empty product container;
c) filling said empty product container with said phases to form a container of said multiphase composition, wherein said filling step comprises transferring predetermined amounts of said compositions in a stream through at least one dispensing means into said empty product container;
d) moving said product container as the multiphase composition is filling said product container to form a non-random pattern; and
e) optionally including a void volume of from about 0% to about 4% in said product container.
